(19) United States
(12) Patent Application Publication
(10) Pub. No.: US 2009/0317364 A1
(43) Pub. Date: Dec. 24, 2009

(54) NOVEL COMPOSITIONS FOR PREVENTION AND TREATMENT OF MASTITIS AND METRITIS

(76) Inventors: Chandrakant Laxminarayan Rathi, Maharashtra (IN); Shilpa Prasanna Risbud, Maharashtra (IN); Bhakti Madhav Manohar, Maharashtra (IN)

Correspondence Address: LADAS & PARRY LLP, 224 SOUTH MICHIGAN AVENUE, SUITE 1600, CHICAGO, IL, 60604, US

(21) Appl. No.: 12/373,104

(22) PCT Filed: Jul. 10, 2007
(86) PCT No.: PCT/IN07/00282

§ 371 Date: Jan. 9, 2009

(30) Foreign Application Priority Data
Jul. 10, 2006 (IN) 1088/MUM/2006

(51) Int. Cl. A61K 45/00; A61K 38/51; A61K 38/43; A61P 25/00

(52) U.S. Cl. 424/93.4; 424/94.5; 424/94.1

(57) ABSTRACT

A novel stable synergistic compositions used for the prevention and/or treatment of mastitis and metritis in mammals comprising a combination of therapeutically effective amount of Serratiopeptidase, Lysozyme, Oscimum sanctum and Azadirechta indica are disclosed in the present invention. The present invention further discloses a method of treatment and/or prevention of an infective condition in a fluid containing organ having a natural exterior orifice, such as the udder of a milk producing animal.

### TECHNICAL FIELD OF THE INVENTION

The present invention relates to novel stable synergistic compositions used for the prevention and/or treatment of mastitis and metritis in mammals comprising a combination of therapeutically effective amount of Serratiopeptidase, Lysozyme, Oscimum sanctum and Azadirechta indica. The present invention further relates to a method of treatment and/or prevention of an infective condition in a fluid containing organ having a natural exterior orifice, such as the udder of a milk producing animal.

### BACKGROUND AND PRIOR ART OF THE INVENTION

Mastitis is an inflammation of the mammary glands of milk-producing animals, for example dairy cows, most often caused by bacterial infection of Streptococcus agalactiae, Staphylococcus aureus, Streptococcus dysgalactiae, Escherichia coli, Klebsiella pneumoniae, Klebsiella oxytoca, Enterobacter aerogenes, Streptococcus uberis, Streptococcus bovis, Streptococcus disgalactiae, Enterococcus faecium, Enterococcus faecalis.

Bacteria enter through the teat canal of the animal and can cause acute, clinical, or sub-clinical mastitis. Over 135 organisms have been documented as causative pathogens for bovine mastitis. Three major groups of pathogens that causes bovine mastitis are gram-positive cocci, gram-negative bacilli and gram-positive bacilli. Hygiene, environmental factors and metabolic disturbances derived from high milk yield combine to create conditions favorable to the onset of mastitis. An increased somatic cell count, associated with mastitis, is positively correlated with infection and negatively correlated with milk production.

Symptoms of mastitis includes inflammation of the mammary glands along with other symptoms like,

Mild signs of flakes or clots in the milk and the animal may have slight swelling of infected quarter.

Severe signs varies from abnormal secretion to hot and swollen quarter or udder; the cow may have fever, rapid pulse, loss of appetite, dehydration and depression; and even death may occur.

Elevated somatic cell count (SCC) of the milk.

Presence of bacteria detected on Bacteriological culturing of milk.

Lowered milk production.

Effects of mastitis on milk Production, milk composition and quality of milk:

Mastitis reduces milk yield and alters milk composition. The magnitude of these changes in individual cow varies with the severity and duration of the infection and the causative microorganisms. Mastitis is almost always caused by bacteria. These microorganisms produce toxins that can directly damage milk-producing tissue of the mammary gland, and the presence of bacteria initiates inflammation within the mammary tissue in an attempt to eliminate the invading microorganisms. The inflammation contributes to decreased milk production and is primarily responsible for the compositional changes observed in milk from infected quarters and cows. In general, compositional changes involve an increase in blood components present in milk and a decrease in normal milk constituents.

Metritis is inflammation of the endometrium (the lining of the uterus), the underlying glandular tissues and the muscular layers. Metritis also causes inflammation of the ovaries.

It is commonly observed that cattle like cows and buffalos suffer their entire life with mastitis and metritis, which ultimately results in their bad health and tremendously decreased milk production. Mastitis is the most hindering disease affecting the economy of the dairy industry, with losses estimated at lacs of rupees annually in the Asian countries alone. As for every clinical case of mastitis, there will be 15 to 40 sub-clinical cases. The majority of these losses are due to reduced milk production.

There are several allopathic as well as synthetic drugs such as Benzyl penicillin, Phenoxymethyl penicillin, Cloxacillin, Nafcillin, Methicillin, Oxacillin, Amoxicillin, Temocillin, Ticarcillin, Indol and Indene acetic acid derivatives, Acetylsalicylic acid derivatives, Fenamates, heteroaryl acetic acid derivatives, propionic acid derivatives, enolic acids para-aminophenol derivatives alkanones nimesulide proquazone known and available to treat the disease.

U.S. Pat. No. 6,414,036 discloses a pharmaceutical composition for treating mastitis wherein oil extract of plants from the Labiatae family are used. The compositions are formulated by combining extracts of essential oils from plants of the Labiatae family with an organic acid or a Group I salt.

CN1390554 discloses an anti-inflammatory paste for treating epidemic parotitis, acute mastitis prepared from fresh cactus, rhubarb natural indigo and sodium sulphate powder.

WO9913892 discloses antimastitic pharmaceutical composition of natural origin comprised of plant extracts for veterinary medical applications in order to treat mastitis in bovine, ovine and caprine animals, and process for obtaining such composition. The composition comprises juice or gel of liliacious plants (Aloe Vera), aqueous extracts of maguey (Agave atrovirens), essential lemon oil (Citrus limon), essential oil of tea tree (Melaleuca alternifolia), comfrey extract (Symphytum officinale). The composition additionally comprises zinc sulphate, sodium salt of ethylene-diamino tetra-acetic acid, citric acid, ascorbic acid and sodium benzoate.

U.S. Pat. No. 3,636,194 discloses compositions for treating mastitis by intramammary infusion, comprising an antibiotic, a vegetable oil, an alcohol-soluble fraction of natural lecithin phospholipids material for promoting dispersion of the oil in milk, the phospholipids being selected from the group consisting of phosphatidylcholine and phosphatidyl ethanolamine and mixtures thereof and present in an amount of at least 0.25% in said oil.

EP1656159 discloses a method of treatment and/or prevention of an infective condition in a fluid-containing organ having a natural exterior orifice, such as the udder of a milk-producing animal or an ear of a subject. This invention also provides a dispersible pharmaceutical composition suitable for infusion into the organ and a process for preparing such a composition. Compositions contain one component from antimicrobial class (antibiotics) in combination with anti-inflammatory analgesic compound like Non Steroidal Anti Inflammatory Drugs (NSAIDs).

GB1181527 discloses a composition for treating mastitis comprising an active substance and a pharmaceutically acceptable oil base. The compositions contain phospholipids material substantially consisting entirely alcohol-soluble material for promoting dispersion of the composition in milk. Specified active agents include penicillin, streptomycin, dihydrostreptomycin, neomycin, polymyxin, tetracyclines, nitrofurazone, cortisone, hydrocortisone, prednisolone, sulpha methazine, sulphamerazine and sulphathiazole.

There are several disadvantages associated with these types of compositions for the treatment of mastitis. All such products are well known to affect negatively on general immunity of subject. It is very commonly observed that the subject looses its immunity after administration of the product. It is also observed that the subject becomes extremely lethargic after administration of the said allopathic or synthetic products. Moreover it is also observed that administration of such product negatively affects milk yield and quality. The cost of these commonly known allopathic products is very high.

Very few antibacterial agents possess anti-inflammatory, anesthetic, antipyretic or analgesic properties in addition to their antibacterial activity. Therefore, treating an infective condition with an antibacterial agent alone typically does not alleviate the inflammation, pain, swelling, fever and other complications that often accompany such an infective condition. These problems are usually not totally resolved until the causal organism of the infective condition has been eliminated or reduced to a sub pathogenic population by the antibacterial agent. The commonly known compositions for treatment of mastitis lack stability and does not provide an extended chemical and/or physical stability. The formulations comprises of pharmaceutically active agent and/or excipient that is prone to oxidative degradation.

Therefore, in view of the aforementioned drawbacks associated with prior art compositions for the treatment of mastitis and metritis, it is apparent that there exists a need for compositions which are effective against microbial infections yet devoid of side effects so as to rejuvenate the general health and immunity of the dairy animals.

### OBJECT OF THE INVENTION

The main object of the present invention is to provide novel stable synergistic compositions used for the treatment of mastitis and metritis comprising therapeutically effective amount of Serratiopeptidase, Lysozyme, Oscimum sanctum and Azadirechta indica having synergistic effect when used in combination.

As per another object, the novel compositions of the present invention are effective at lower doses of the active agent providing targeted delivery of the active agent to the site of infection with minimal/no irritation upon administration and minimal/no side effects in comparison to the synthetic and allopathic drugs used for the treatment of mastitis and metritis.

As per yet another object of the invention, the novel compositions rejuvenates the general health and immunity of the subject naturally on administration and are effective against a wide variety of infectious organisms and inflammatory and infectious components like pain, inflammation, fever, edema.

Further object of the present invention is to provide novel compositions having economic significance in comparison to the commonly available allopathic and synthetic drugs.

Still further object of the invention is to provide a method of treatment and/or prevention of an infective condition in a fluid containing organ having a natural exterior orifice, such as the udder of a milk producing animal.

### SUMMARY OF THE INVENTION

The present invention discloses novel stable synergistic compositions used for the treatment of mastitis and metritis in mammals comprising combination of therapeutically effective amount of Serratiopeptidase, Lysozyme, Oscimum sanctum and Azadirechta indica. The present invention further discloses a novel method of treatment and/or prevention of an infective condition in a fluid containing organ having a natural exterior orifice, such as the udder of a milk producing animal. The present invention still further discloses evaluation of the antibacterial properties of the novel compositions on a wide variety of causative organisms.

### DESCRIPTION OF THE INVENTION

The present invention describes novel synergistic stable compositions for the treatment of mastitis and metritis in mammals.

The novel composition as per the present invention comprises of a combination of absolutely Natural products such as Serratiopeptidase, Lysozyme (Muramidase), Oscimum sanctum and Azadirechta indica.

The novel compositions of the present invention have minimal or no side effects and also have economic significance in comparison to the commonly known compositions.

The present invention further relates to a method of treatment and/or prevention of an infective condition in a fluid containing organ having a natural exterior orifice, such as the udder of a milk producing animal. The novel method of treatment for mastitis and metritis comprises of administering a natural combination of Serratiopeptidase, Lysozyme(Muramidase), Oscimum sanctum and Azadirechta indica, as an antibacterial and anti-inflammatory agent through oral and/or topical route.

Described below are the ingredients and qualities of the natural products used in the composition for the treatment of mastitis and metritis.

1] Serratiopeptidase:

Serratiopeptidase also known as Serrapeptase is a proteolytic enzyme that stimulates immunity, reduces edema, and fights inflammation. Serratiopeptidase is isolated from the non-pathogenic Enterobacteria Serratia E15. The enzyme is found naturally in the intestine of the silkworm, which is used by the silkworm to dissolve the cocoon and emerge as a moth. When consumed as uncoated tablets or capsules, the enzyme is destroyed by the acid in the stomach. However, when enteric coated, the enzyme passes through the stomach unaffected and get absorbed in the intestine.

Serrapeptase when given in combination with antimicrobial agents delivers increased concentrations of the antimicrobial agent to the site of infection. The mechanism of antibacterial action of serratiopeptidase can be explained, as an inhibitor of biofilm formation of bacterial cell wall. Bacteria often endure a process called biofilm formation, which results in resistance to antimicrobial agents.

| Chemical characterization | Microbial fermentation enzyme |
|---|---|
| of active component | preparation from bacterial culture. |
| Source/Origin | Serratia spp. |
| Systematic Name (IUB) | Serratiopeptidase |
| IUB/CAS number | CAS 37312-62-2 |
| Hazardous ingredients | None |
| Classification | Enzyme protease |
| Pharmacopoeial Status | Martindale Pg no 1662 |

2] Lysozyme:

Lysozyme is also known as Muramidase and it is isolated from the extracts of purified chicken egg white along with naturally occurring biologically active proteins. Lysozyme acts as a “natural” antibacterial. The therapeutic effectiveness of lysozyme is actually based on its ability to control the growth of susceptible bacteria and to modulate host immunity against infections. The ability to control the growth of the susceptible bacteria is due to the biological activity of the enzyme. Antibiotic activity and immune stimulating effects of lysozyme impart therapeutic benefits.

Lysozyme hydrolyzes preferentially the β-1,4 glucosidic linkages between N-acetylmuramic acid and N-acetylglucosamine which occur in the mucopeptide cell wall structure of certain microorganisms, such as Micrococcus lysodeikticus. A somewhat more limited activity is exhibited towards chitin oligomers. Lysozyme is of widespread distribution in animals and plants. Lysozyme is also found in mammalian secretions and tissues, saliva, tears, milk, cervical mucus, leucocytes, kidneys, etc

| Chemical characterization | |
|---|---|
| of active component | Enzyme preparation from animal origin. |
| Source/Origin | Extracts of purified chicken egg white |
| Systematic Name (IUB) | Peptidoglycan N-acetylmuramoylhydrolase |
| IUB/CAS number 5 | CAS 3.2.1.17 |
| Hazardous ingredients | None |
| Classification | Own Antibiotic |
| Pharmacopoeial Status | Martindale Pg no 1638 |

3] Oscimum sanctum:

Oscimum sanctum acts as a COX-2 inhibitor and provides the benefits of an analgesic owing to active constituent Eugenol (1-hydroxy-2-methoxy-4-allylbenzene). Studies have shown Oscimum sanctum to be effective for the treatment of diabetes, as it reduces the blood glucose levels and this benefit is due to its antioxidant properties. The same study showed that there is a significant reduction in total cholesterol levels with Oscimum sanctum.

Oscimum sanctum extracts are used for common colds, headaches, stomach disorders, inflammation, heart disease, various forms of poisoning, and malaria. Oscimum sanctum can be consumed in various forms like herbal tea, dried powder, fresh leaf, or mixed with ghee. Essential oil extracted from Karpoora Oscimum sanctum is mostly used for medicinal purposes and in herbal toiletry. The dried leaves of Oscimum sanctum being an excellent insect repellant are mixed with stored grains to repel insects.

4] Azadirechta indica

Azadirechta indica plant has numerous medicinal properties hence used for various conditions like digestive disorders, diabetes, high cholesterol, cancer, etc.

Azadirechta indica has anti malarial properties hence used for the treatment of malaria. Oil of Azadirechta indica is used extensively by the cosmetic industry for the preparation of cosmetics like soap, shampoo, balms and creams. All parts of the tree (seeds, leaves, flowers and bark) are used for preparing many different medical preparations. Azadirechta indica twigs are used for brushing teeth in India-perhaps one of the earliest and most effective forms of dental care. In some parts of Sub-Saharan Africa, the bark is used as both toothbrush and toothpaste. Azadirechta indica tree is of great importance for its anti-desertification properties and possibly as a good carbon dioxide sink.

The primary interest of research scientists is the insecticidal activity of Azadirechta indica. The secondary metabolites of various trees have biological activity, but azadirachtin of Azadirechta indica has utmost ecological importance. Studies have shown wide spectrum of insecticidal activity for Azadirechta indica and the numerous species affected. Azadirechta indica acts by breaking the insect's lifecycle. Research has increased in the past few years as the desire for safer pest control methods increases and it becomes apparent that this tree will be able to play a role in integrated pest management systems.

Azadirechta indica is deemed very effective in the treatment of scabies although only preliminary scientific proof exists which still has to be corroborated, and is recommended for those who are sensitive to permethrin, a known insecticide which might be irritant. Also, the scabies mite has yet to become resistant to Azadirechta indica, so in persistent cases Azadirechta indica has been shown to be very effective. There is also anecdotal evidence of its effectiveness in treating infestations of head lice in humans.

In view of the above properties of the individual substances, the present inventors have developed novel compositions from natural product for the treatment of mastitis and metritis in mammals. The novel stable compositions containing the combination of therapeutically effective amount of Serratiopeptidase, Lysozyme, Oscimum sanctum and Azadirechta indica provides synergistic activity.

The present invention is more specifically explained by following examples. However, it should be understood that that the scope of the present invention is not limited by the examples in any manner. It will be appreciated by any person skilled in this art that the present invention includes the following examples and further can be modified and altered within the technical concept of the present invention.

### Examples

### Example 1

| | | | | | | | Stability | Stability |
|---|---|---|---|---|---|---|---|---|
| | | | | | | | for 1 | for 6 |
| Sr. | C1 % | C2 % | C3 % | C4 % | C5 % | C6 % | month | months |
| 1 | 1 | 10 | 10 | 10 | 10 | Q.S. | Stable | Stable |
| 2 | 2 | 10 | 10 | 10 | 10 | Q.S. | Stable | Stable |
| 3 | 3 | 10 | 10 | 10 | 10 | Q.S. | Stable | Stable |
| 4 | 4 | 10 | 10 | 10 | 10 | Q.S. | Stable | Stable |
| 5 | 5 | 10 | 10 | 10 | 10 | Q.S. | Stable | Stable |

| | |
|---|---|
| C1 = Serratiopeptidase | 1-5% |
| C2 = Lysozyme | 10% |
| C3 = Oscimum sanctum | 10% |
| C4 = Azadirechta indica | 10% |
| C5 = Citric acid | 10% |
| C6 = Malt dextrin | Q.S. |

### Example 2

| | | | | | | | Stability | Stability |
|---|---|---|---|---|---|---|---|---|
| | | | | | | | for 1 | for 6 |
| Sr. | C1 % | C2 % | C3 % | C4 % | C5 % | C6 % | month | months |
| 1 | 2 | 10 | 10 | 10 | 10 | Q.S. | Stable | Stable |
| 2 | 2 | 15 | 10 | 10 | 10 | Q.S. | Stable | Stable |
| 3 | 2 | 20 | 10 | 10 | 10 | Q.S. | Stable | Stable |
| 4 | 2 | 25 | 10 | 10 | 10 | Q.S. | Stable | Stable |
| 5 | 2 | 30 | 10 | 10 | 10 | Q.S. | Stable | Stable |

| | |
|---|---|
| C1 = Serratiopeptidase | 2% |
| C2 = Lysozyme | 10-30% |
| C3 = Oscimum sanctum | 10% |
| C4 = Azadirechta indica | 10% |
| C5 = Citric acid | 10% |
| C6 = Malt dextrin | Q.S. |

### Example 3

| | | | | | | | Stability | Stability |
|---|---|---|---|---|---|---|---|---|
| | | | | | | | for 1 | for 6 |
| Sr. | C1 % | C2 % | C3 % | C4 % | C5 % | C6 % | month | months |
| 1 | 2 | 20 | 10 | 10 | 10 | Q.S. | Stable | Stable |
| 2 | 2 | 20 | 15 | 10 | 10 | Q.S. | Stable | Stable |
| 3 | 2 | 20 | 20 | 10 | 10 | Q.S. | Stable | Stable |
| 4 | 2 | 20 | 25 | 10 | 10 | Q.S. | Stable | Stable |
| 5 | 2 | 20 | 30 | 10 | 10 | Q.S. | Stable | Stable |

| | |
|---|---|
| C1 = Serratiopeptidase | 2% |
| C2 = Lysozyme | 20% |
| C3 = Oscimum sanctum | 10-30% |
| C4 = Azadirechta indica | 10% |
| C5 = Citric acid | 10% |
| C6 = Malt dextrin | Q.S. |

### Example 4

| | | | | | | | Stability | Stability |
|---|---|---|---|---|---|---|---|---|
| | | | | | | | for 1 | for 6 |
| Sr. | C1 % | C2 % | C3 % | C4 % | C5 % | C6 % | month | months |
| 1 | 2 | 20 | 20 | 10 | 10 | Q.S. | Stable | Stable |
| 2 | 2 | 20 | 20 | 15 | 10 | Q.S. | Stable | Stable |
| 3 | 2 | 20 | 20 | 20 | 10 | Q.S. | Stable | Stable |
| 4 | 2 | 20 | 20 | 25 | 10 | Q.S. | Stable | Stable |
| 5 | 2 | 20 | 20 | 30 | 10 | Q.S. | Stable | Stable |

| | |
|---|---|
| C1 = Serratiopeptidase | 2% |
| C2 = Lysozyme | 20% |
| C3 = Oscimum sanctum | 20% |
| C4 = Azadirechta indica | 10-30% |
| C5 = Citric acid | 10% |
| C6 = Malt dextrin | Q.S. |

### Example 5

| | | | | | | | Stability | Stability |
|---|---|---|---|---|---|---|---|---|
| | | | | | | | for 1 | for 6 |
| Sr. | C1 % | C2 % | C3 % | C4 % | C5 % | C6 % | month | months |
| 1 | 2 | 20 | 20 | 10 | 10 | Q.S. | Unstable | Unstable |
| 2 | 2 | 20 | 20 | 15 | 10 | Q.S. | Unstable | Unstable |
| 3 | 2 | 20 | 20 | 20 | 10 | Q.S. | Unstable | Unstable |
| 4 | 2 | 20 | 20 | 25 | 10 | Q.S. | Unstable | Unstable |
| 5 | 2 | 20 | 20 | 30 | 10 | Q.S. | Unstable | Unstable |

| | |
|---|---|
| C1 = Serratiopeptidase | 2% |
| C2 = Lysozyme | 20% |
| C3 = Oscimum sanctum | 20% |
| C4 = Azadirechta indica | 20% |
| C5 = Citric acid | 10% |
| C6 = Zeolite | Q.S. |

### Example 6

| | | | | | | | Stability | Stability |
|---|---|---|---|---|---|---|---|---|
| | | | | | | | for 1 | for 6 |
| Sr. | C1 % | C2 % | C3 % | C4 % | C5 % | C6 % | month | months |
| 1 | 2 | 20 | 20 | 20 | 10 | Q.S. | Stable | Stable |
| 2 | 2 | 20 | 20 | 20 | 10 | Q.S. | Stable | Stable |
| 3 | 2 | 20 | 20 | 20 | 10 | Q.S. | Stable | Stable |
| 4 | 2 | 20 | 20 | 20 | 10 | Q.S. | Stable | Stable |
| 5 | 2 | 20 | 20 | 20 | 10 | Q.S. | Stable | Stable |

| | |
|---|---|
| C1 = Serratiopeptidase | 2% |
| C2 = Lysozyme | 20% |
| C3 = Oscimum sanctum | 20% |
| C4 = Azadirechta indica | 20% |
| C5 = Citric acid | 10% |
| C6 = DCP | Q.S. |

### Example 7

| | | | | | | | | Stability | Stability |
|---|---|---|---|---|---|---|---|---|---|
| Sr. | C1 % | C2 % | C3 % | C4 % | C5 % | C6 % | C7 % | for 1 | for 6 |
| 1 | 2 | 20 | 20 | 20 | 10 | Q.S. | 5 | Stable | Stable |
| 2 | 2 | 20 | 20 | 20 | 10 | Q.S. | 5 | Stable | Stable |
| 3 | 2 | 20 | 20 | 20 | 10 | Q.S. | 5 | Stable | Stable |
| 4 | 2 | 20 | 20 | 20 | 10 | Q.S. | 5 | Stable | Stable |
| 5 | 2 | 20 | 20 | 20 | 10 | Q.S. | 5 | Stable | Stable |

| | |
|---|---|
| C1 = Serratiopeptidase | 2% |
| C2 = Lysozyme | 20% |
| C3 = Oscimum sanctum | 20% |
| C4 = Azadirechta indica | 20% |
| C5 = Citric acid | 10% |
| C6 = Malt dextrin | Q.S. |
| C7 = Saccharomyces boulardii. | 5% |

### Example 8

### Evaluation of the Antibacterial Properties

a] Testing with a Gram positive organism:

Formulation: Polyenzyme formulations.

Organisms used for the test: Streptococcus spp., Staphylococcus spp. Klebsiella spp. Enterococcus spp. E. coli.

Method: Drop inoculation (20 mg/ml) on lawn culture.

Procedure: The test organism was inoculated into nutrient broth and incubated overnight.

This inoculum was matched with 0.5 Macfarland standards and then inoculated onto respective Agar plates. A penicillin disk (potency 10 units; HiMedia Laboratories, Mumbai) was placed onto the plate as a control. Drops (20 μl) of various concentrations of polyenzyme formulations, and components were applied separately onto the inoculated plates. The plates were incubated at 37° C. for 24 hours.

Polyenzyme formulations produced a zone of clearance on the lawn culture plate.

b]Lab Trial Results

| | | Growth | | | Growth |
|---|---|---|---|---|---|
| Example | | 2.5 | Growth | Growth | 15 |
| Trial | Causative organism | mg/ml | 5 mg/ml | 10 mg/ml | mg/ml |
| E2 T1 | E. coli | +++ | ++ | ± | — |
| | S. aureus | +++ | + | ± | — |
| | K. pneumoniae | +++ | ++ | + | — |
| | Streptococcus spp. | +++ | + | ± | — |
| E2 T2 | E. coli | +++ | ± | ± | — |
| | S. aureus | +++ | ± | ± | — |
| | K. pneumoniae | +++ | + | ± | — |
| | Streptococcus spp. | +++ | + | ± | — |
| E2 T3 | E. coli | +++ | ± | — | — |
| | S. aureus | +++ | ± | — | — |
| | K. pneumoniae | +++ | ± | ± | — |
| | Streptococcus spp. | +++ | ± | ± | — |
| E2 T4 | E. coli | +++ | — | — | — |
| | S. aureus | +++ | — | — | — |
| | K. pneumoniae | +++ | — | — | — |
| | Streptococcus spp. | +++ | — | — | — |
| E3 T3 | E. coli | +++ | ± | — | — |
| | S. aureus | +++ | ± | — | — |
| | K. pneumoniae | +++ | ± | ± | — |
| | Streptococcus spp. | +++ | ± | ± | — |
| E4 T3 | E. coli | +++ | ± | — | — |
| | S. aureus | +++ | ± | — | — |
| | K. pneumoniae | +++ | ± | ± | — |
| | Streptococcus spp. | +++ | ± | ± | — |
| E6 T3 | E. coli | +++ | ± | — | — |
| | S. aureus | +++ | ± | — | — |
| | K. pneumoniae | +++ | ± | ± | — |
| | Streptococcus spp. | +++ | ± | ± | — |

Wherein,

E 2 T1 refers to trial 1 with the composition disclosed in example 2;

E2 T2 refers to trial 2 with the composition disclosed in example 2;

E2 T3; refers to trial 3 with the composition disclosed in example 2;

E2 T4; refers to trial 4 with the composition disclosed in example 2;

E3 T3; refers to trial 3 with the composition disclosed in example 3;

E4 T3; refers to trial 3 with the composition disclosed in example 4;

E5 T3; refers to trial 3 with the composition disclosed in example 2;

E6 T3; refers to trial 3 with the composition disclosed in example 6;

On the basis of these trials, the final composition was finalized and used for the further trials i.e. in trial 2a and 2b.

### Example 9

a) Lab trials 2a (in vitro) for synergy.

### Evaluation of Synergy

| | Inhibition zone diameter In |
|---|---|
| | cm for Staphylococcus. |
| Components added in wells for diffusion. | aureus |
| Control | Lawn growth |
| Serratiopeptidase | 0.5 |
| Lysozyme | 1.15 |
| Oscimum | 0.1 |
| Azadirechta | 0.2 |
| Serratiopeptidase + Lysozyme | 1.95 |
| Serratiopeptidase + Oscimum | 0.45 |
| Serratiopeptidase + Azadirechta | 0.56 |
| Lysozyme + Oscimum | 1.2 |
| Lysozyme + Azadirechta | 1.25 |
| Oscimum + Azadirechta | 0.2 |
| Serratiopeptidase + Lysozyme + Oscimum | 1.29 |
| Serratiopeptidase + Lysozyme + Azadirechta | 1.35 |
| Lysozyme + Oscimum + Azadirechta | 1.2 |
| Serratiopeptidase + Oscimum + Azadirechta | 0.7 |
| Serratiopeptidase + Lysozyme + | 2.5 |
| Oscimum + Azadirechta | |

b) Lab trials 2b (in vitro) for synergy.

| | Inhibition zone In diameter |
|---|---|
| | cm for Staphylococcus. |
| Components added on top of the culture. | aureus |
| Control | Lawn growth. |
| Serratiopeptidase | 0.61 |
| Lysozyme | 1.1 |
| Serratiopeptidase + Lysozyme | 2.03 |

### Example 10

### Effect of Said Formulation in Sub Clinical Mastitis Cases

Studies were conducted to evaluate the efficacy of said stable composition. The formulation was assessed using in vitro and in vivo studies.

a) In Vitro Study:

Milk samples from mastitis cases were collected and the causative organism in particular case was identified by staining and observing the morphological characteristics.

The test drug was assessed for its in-vitro antibacterial efficacy against the isolated mastitis causing organism using disc diffusion techniques. Blank sensitivity discs of 6.25 mm diameter were punched from Whatman filter paper and sterilized by dry heat. The blank discs were weighed several times to get the exact weight of blank disc. The mean weight of one disc so obtained was 3.08 mg. The blank discs were separately impregnated with the aqueous solution of the formulation as described below.

The aqueous solution of the test formulation was taken in a tuberculin syringe and then added drop by drop on each disc. After drying, the process was repeated thrice. The discs were then weighed to know the exact weight of the test drug in each disc. The sensitivity discs so prepared were assessed for the antibacterial efficacy.

Results

All the animals were observed to be normal and healthy. But the presence of staphylococcus organisms in the milk samples and the CMT revealed sub-clinical mastitis. The results of assessment of the antibacterial activity of the formulation “test formulation” by disc diffusion technique are shown in table 1.

TABLE 1
Antibacterial activity of “Test formulation” against staphylococcus

| | | Weight of test | Average zone of |
|---|---|---|---|
| Weight of blank disc | Weight of discs after | drug Test | inhibition for 20 trials |
| (mg) | impregnation (mg) | formulation (mg) | diameter (mm) |
| 3.08 ± 0.01 | 4.20 ± 0.02 | 1.122 ± 0.01 | 16.00 ± 0.13 |

The zone of inhibition (diameter in mm) of size 16.00±0.13 was observed indicative of positive effect of Test formulation on mastitis causing bacteria staphylococcus.

In the in vivo studies the bacterial colony count on day 1^{st} of the experiment was observed to be 228.43±13.37. However reduction in the bacterial load was observed from 3^{rd} day onwards (175.23±11.16 on day 3^{rd}) indicative of positive effect of the drug. However, no bacterial colonies were observed in the milk sample of 7^{th} day (Table 2).

TABLE 2
Antibacterial efficacy of “Test formulation” in in-vivo studies

| Day of experiment | Mean Colony count ± S.E. |
|---|---|
| 1 | 228.43 ± 13.37 |
| 2 | 202.02 ± 13.11 |
| 3 | 175.23 ± 11.16 |
| 4 | 75.23 ± 9.08 |
| 5 | 65.78 ± 9.11 |
| 6 | 15.00 ± 6.90 |
| 7 | 0 |

The present investigation was concluded as follows:
The drug “Test formulation” is effective in controlling the bacterial load in sub-clinical mastitis cases in buffaloes.
The drug “Test formulation” is effective in inhibiting the bacterial colonies of staphylococcus organisms isolated from sub-clinical mastitis cases in buffaloes.

b) In Vivo Study

Twenty she buffaloes suffering from sub-clinical mastitis were selected by conducting the California mastitis test (CMT) and processing the milk samples in laboratory for presence of bacteria.

The drug was applied daily on the udder (external application) for a period of 07 days. Daily milk samples from the animals were collected and processed in the lab for bacterial load. Efficacy against mastitis was judged on the basis of clinical signs (if any) bacterial load, somatic cell count and the time required (in days) for the reduction in the bacterial count.

### Example 11

### Effect of Said Formulation in Clinical Metritis Cases in Animals

a) In Vitro Study

Uterine swab samples from metritis cases (06 cases) were collected and the causative organism was identified to be streptococcus and staphylococcus as mixed infection using bacteriological parameters.

The test drug was assessed for its in-vitro antibacterial efficacy against the isolated metritis causing organisms using disc diffusion techniques. The blank sensitivity discs of 6.25 mm diameter were punched from Whatman filter paper and sterilized by dry heat. The blank discs were weighed several times to get the exact weight of blank disc. The mean weight of one disc so obtained was 3.08 mg. The blank discs separately impregnated with the aqueous solution of the formulation “Test formulation” as described below.

The aqueous solution of the formulation “Test formulation” was taken in a tuberculin syringe and added drop by drop on each disc. After drying, the process was repeated thrice. The discs were then weighed to know the exact weight of the test drug in each disc. The sensitivity discs so prepared were assessed for the antibacterial efficacy.

Results:

The results of assessment of the antibacterial activity of the formulation “Test formulation” by disc diffusion technique are shown in table 1.

TABLE 1
Antibacterial activity of “Test formulation” against staphylococcus

| | | Weight of test | Average zone of |
|---|---|---|---|
| Weight of blank disc | Weight of discs after | drug Test | inhibition for 20 trials |
| (mg) | impregnation (mg) | formulation (mg) | diameter (mm) |
| 3.08 ± 0.01 | 4.39 ± 0.06 | 1.31 ± 0.12 | 9.26 ± 0.10 |

The zone of inhibition (diameter in mm) of size 9.26±0.10 were observed indicative of positive effect of Test formulation on metritis causing bacteria such as streptococcus and staphylococcus.

In the in vivo studies the bacterial colony count on day 1^{st} of the experiment was observed to be 326.7±16.11. However reduction in the bacterial load was observed from 5^{th} day onwards (168.16±9.28 on day 5^{th}) indicative of positive effect of the drug. However, 104.88±6.78 bacterial colonies were observed in the uterine swab samples of 7^{th} day (Table 2). The animals were then shifted to antibiotic treatment for further recovery.

TABLE 2
Antibacterial efficacy of Test formulation” in in-vivo studies

| Day of experiment | Mean Colony count ± S.E. |
|---|---|
| 1 | 326.7 ± 16.11 |
| 2 | 320.32 ± 15.98 |
| 3 | 278.76 ± 15.16 |
| 4 | 200.03 ± 10.79 |
| 5 | 166.34 ± 10.28 |
| 6 | 127.57 ± 8.79 |
| 7 | 104.88 ± 6.78 |

The present investigation was concluded as follows:
The drug “Test formulation” is effective in controlling the bacterial load in clinical metritis cases in cattle.
The drug “Test formulation” is effective in inhibiting the bacterial colonies of streptococcus and staphylococcus organisms isolated from clinical metritis cases in cattle.
Test formulation can be used to minimize the use of antibiotics in metritis cases.

b) In Vivo Study:

The poly enzyme formulation “Test formulation” was assessed for its antibacterial efficacy in six cows suffering from the metritis. All the animals were examined for the clinical signs and symptoms and the metritis was confirmed.

Uterine swab samples were collected by taking all aseptic precautions and were processed in laboratory to confirm bacterial infection.

The animals selected for the present study were treated with the test drug. Test formulation was dissolved in sterile distilled water and administered intra-uterine at the dose rate of 5 gm per day (in 20 ml sterile distilled water) for a period of 7 days. Daily uterine swab samples from the affected animals were collected and processed in the lab for bacterial load/count.

The swabs were processed in the nutrient broth and bacteria were isolated by observing their growth on nutrient agar. Mixed infection of streptococcus and staphylococcus was diagnosed in the affected animals.

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

We claim:

1. A stable synergistic composition comprising antimicrobial and anti-inflammatory enzymes and plant extracts for prevention and treatment of mastitis and/or metritis in milk producing animals, optionally with a probiotic.

2. The composition as claimed in claim 1, wherein the antimicrobial and antiinflammatory enzymes selected are Serratiopeptidase and Lysozymes

3. The composition as claimed in claim 1, wherein the plant extracts selected are the group consisting of the Oscium sanctum and Azardirecta indica.

4. The composition as claimed in claim 1, wherein the said composition comprises Serratiopeptidase, Lysozymes, Oscium sanctum and Azadirecta indica.

5. The composition as claimed in claim 1, wherein the said composition comprises Serratiopeptidase at the concentration of 05 to 200 mg of the total formulation and Lysozymes at the concentration of 10 to 1000 mg of the total formulation.

6. The composition as claimed in claim 1, wherein the said composition comprises Oscium sanctum extracts at the concentration of 1000 to 2000 mg of the total formulation and Azadirecta indica extracts at the concentration of 1000 to 3000 mg of the total formulation.

7. The composition as claimed in claim 1, wherein the probiotic is Saccharomyces boulardii present at the concentration of 5% of the total formulation.

8. The composition as claimed in claim 1, wherein the said composition is effective against a group of bacteria selected from streptococcus agalactiae, staphylococcus aureus, streptococcus dysgalactiae, Escherichia coli, klebsielle pnemoniae, klebsielle oxytoca, enterobacter aerogenes, streptococcus uberis, streptococcus bovis, streptococcus disgalactiae, enterococcus faecium, enterococcus faecalis.

9. The composition according to claim 1 is applied topically as an aqueous solution without any carrier base wherein the disease is mastitis.

10. The composition according to claim 1 is applied intrauterine infusion as an aqueous solution wherein the disease is metritis

11. A stable synergistic composition according to any of the preceding claims comprising serratiopeptidase in the range of 75 to 150 mg; lysozyme in the range of 750 to 1000 mg; Oscimum sanctum in the range of 1000 to 1500 mg and Azadirechta indica in the range of 1000 to 1500 mg.
